(19) United States
(12) Patent Application Publication
(10) Pub. No.: US 2012/0315236 A1
(43) Pub. Date: Dec. 13, 2012

(54) AGENTS FOR OXIDATIVELY CHANGING THE COLOUR OF KERATIN-CONTAINING FIBRES

(76) Inventors: Konstantin Goutsis, Erkelenz (DE); Frank Janssen, Neuss (DE); Ina Franke, Duesseldorf (DE)

(21) Appl. No.: 13/566,505

(22) Filed: Aug. 3, 2012

Related U.S. Application Data
(63) Continuation of application No. PCT/EP2011/065602, filed on Sep. 9, 2011.

(30) Foreign Application Priority Data
Oct. 11, 2010 (DE) 102010042252.5

(51) Int. Cl. A61K 8/23; A61Q 5/08; A61K 8/22

(52) U.S. Cl. 424/62

(57) ABSTRACT

Agents for lightening keratinic fibers include at least two preparations (A) and (B) packaged separately from one another, as well as optionally a further preparation (C) packaged separately from (A) and (B), which are mixed immediately before utilization to yield a utilization mixture, wherein preparation (A) contains at least one persulfate, and the preparations (B) are flowable and contain at least one oxidizing agent, and where preparations (B) and/or preparations (C) further contain(s) at least one natural polymer.

### CROSS-REFERENCES TO RELATED APPLICATIONS

This application is a continuation of PCT/EP2011/065602, filed on Sep. 9, 2011, and further claims priority under 35 U.S.C. §119 to DE 10 2010 042 252.5 filed on Oct. 11, 2010, both of which are hereby incorporated by reference.

### FIELD OF THE INVENTION

The present invention generally relates to agents for oxidatively changing color in the cosmetic sector, which are particularly suitable for lightening keratinic fibers, in particular human hair.

### BACKGROUND OF THE INVENTION

Changing the shape and color of hair represents an important sector of modern cosmetology. In addition to coloring, lightening of the inherent hair color, or hair-bleaching, is a very specific desire of many consumers since a blonde hair color is regarded as attractive and desirable in terms of fashion. A variety of hair-bleaching agents, with differing hair-bleaching performance, are available on the market for this purpose.

The oxidizing agents contained in hair-bleaching agents are capable of lightening hair fibers by oxidative destruction of the hair's own melanin dye. For a moderate hair-bleaching effect it is sufficient to use hydrogen peroxide, optionally with the use of ammonia or other alkalizing agents, as the only oxidizing agent; to achieve a stronger hair-bleaching effect it is usual to use a mixture of hydrogen peroxide and peroxodisulfate salts and/or peroxomonosulfate salts.

For stability reasons, commercially conventional hair-bleaching agents are usually offered in two preparations packaged separately from one another, which are mixed together immediately before use to yield a completed utilization preparation. Commercially usual hair-bleaching agents are usually made up of a liquid oxidizing agent preparation and a powder that contains solid oxidizing agents. Products having further components are likewise offered commercially. Good miscibility of the various preparations is essential for problem-free handling of the ready-to-use hair-bleaching agent. Clumping means that only a portion of the active substances used can reach the fibers, and results in a weakening of the desired hair-bleaching effect. In addition, incomplete homogenization produces fluctuations in the concentrations of the various active substances in the completed utilization preparation, and consequently an inhomogeneous hair-bleaching effect on the fibers. A need therefore still exists for novel hair-bleaching-agent preparations having good mutual miscibility and improved hair-bleaching results.

The viscosity of the preparation furthermore plays an important role. A good ability for the preparation to be distributed over the entire length, and prevention of dripping, are essential properties for optimal handling of the decolorizing agent. The customer expects in this context that the agent will be viscous enough, upon application and during the contact time, so as not to drip onto material in the vicinity (e.g. clothing or floors), which can cause undesired discoloration. At the same time, however, sufficient flowability of the agent must be ensured so as to enable homogeneous application and good distribution of the agent over the entire hair length. The object that presents itself is that of adapting the flowability of the agent to consumers' demands without degrading the stability of the active substances of the agent, e.g. hydrogen peroxide. In many cases, hair-bleaching agents that contain emulsions are used. The rheological properties of emulsions tend to change during storage as a function of the storage conditions, thus influencing the aforementioned distribution and mixing capabilities. Gels, on the other hand, exhibit better homogeneity and longer shelf stability. A need therefore continues to exist for novel hair-bleaching preparations having good stability and improved viscosity.

A further important aspect of the utilization of hair-bleaching agents is monitoring of the lightening operation on the fibers. The decolorizing operation is usually monitored by the consumer at least once during the contact time. Commercially usual hair-bleaching agents are, in the ready-to-use state, usually white or colored cloudy gels or emulsions. When these hair-bleaching agents are utilized, it is necessary to remove the agent in one or more regions of the fibers in order to monitor the decolorizing process during the contact time. The consumer can thereby assess the progress of the color change. If necessary, the utilization mixture must then be reapplied onto the corresponding sites on the fibers in order to continue the lightening process. The operation must be repeated, as applicable, for further monitoring. The use of transparent utilization mixtures considerably simplifies this monitoring step. Removal of the hair-bleaching agent from the fibers is not necessary. Instead, the transparency of the utilization mixture allows a direct visual assessment of the decolorizing operation at any desired point in time during the contact period. The provision of transparent hair-bleaching agents consequently results in improved handling of the hair-bleaching agent and in simplified utilization.

WO 2005/067874 A1 describes hair-bleaching agents that contain a mixture of an oxidizing agent, at least one stabilizer, at least one polymer thickener, and water or an aqueous solvent. “Transparency” and “thickened consistency” are indicated according to this invention as desirable properties of the agent (p. 2, paragraphs 1 and 2). Polymer thickeners made of synthetic polymers and alkali magnesium silicates are claimed in WO 2005/067874 A1. Example 13 (p. 14) of the aforesaid Application describes a hair-bleaching agent that contains, inter alia, xanthan gum alongside an acrylic acid polymer in the powdered component. Xanthan gum appears, however, to have been inserted at random at this juncture. Neither the description nor the claimed invention of the aforesaid Application allow the conclusion that the properties of xanthan gum are relevant to the invention, and the advantages of using natural polymers of these agents were certainly not recognized.

Accordingly, it is desirable to improve the properties of commercially usual hair-bleaching agents in terms of their miscibility and viscosity. In addition, it is desirable to obtain a homogeneous, transparent hair-bleaching utilization mixture having those properties, in order additionally to enable visual monitoring of the lightening operation without removing the preparation from the fibers.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

Agents for lightening keratinic fibers, containing at least two preparations (A) and (B) packaged separately from one another, as well as optionally a further preparation (C) packaged separately from (A) and (B), which are mixed immediately before utilization to yield a utilization mixture, where preparation (A) contains at least one persulfate, and preparation (B) is flowable and contains at least one oxidizing agent, wherein preparation (B) and/or preparation (C) further contain(s) at least one natural polymer.

A method for changing the color of keratinic fibers, wherein at least two preparations (A) and (B) packaged separately from one another, of which preparation (A) contains at least one persulfate and preparation (B) at least one oxidizing agent and at least one natural polymer, are mixed to yield a utilization mixture, the latter is applied onto the fibers, and is rinsed out again after a contact time.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

It has become apparent that the addition of a natural polymer to the oxidizing agent preparation results in a flowable preparation that allows easy incorporation of a powdered preparation and good homogenization of the two components to yield a completely mixed utilization mixture. In addition, the preparation according to the present invention exhibits good properties in terms of viscosity. A further advantage of the hair-bleaching agent according to the present invention is its transparency, which enables visual monitoring of the degree of lightening of the keratinic fibers during the lightening process.

Agents for lightening keratinic fibers, containing at least two preparations (A) and (B) packaged separately from one another, as well as optionally a further preparation (C) packaged separately from (A) and (B), which are mixed immediately before utilization to yield a utilization mixture, where
i. preparation (A) contains at least one persulfate, and
ii. preparation (B) is flowable and contains at least one oxidizing agent,
   wherein preparation (B) and/or preparation (C) further contain(s) at least one natural polymer.

“Keratinic fibers” or also “keratin fibers” are to be understood in this context as firs, wool, feathers, and in particular human hair. Although the agents according to the present invention are suitable principally for lightening keratin-containing fibers, nothing in principle conflicts with use in other sectors as well.

Preparations (A) are preferably powdered. Powders made up of solid constituents having different particle sizes can be used. It can usually be preferred, however, if the powders exhibit the most homogeneous possible particle size, in particular in order to facilitate uniform dispersion resp. dissolution of the powders in preparations (B).

Preparations (A) can contain the active substances in a solid cosmetic carrier. A solid cosmetic carrier can contain salts of silicic acid, in particular salts of silicates and metasilicates with ammonium, alkali metals, and alkaline earth metals. Metasilicates in particular, which in accordance with the formula (SiO₂)ₙ(M₂O)ₘ, where M denotes an ammonium ion, alkali metal, or half a stoichiometric equivalent of an alkaline earth metal, are notable for a ratio between n and m of ≦1 and can be construed as a chain-like polymeric structure of the [SiO₃]²⁻ anion, can be used with preference. Sodium metasilicate of the formula [Na₂SiO_{∞}], is particularly preferred in this context. Likewise preferred according to the present invention are those silicates that are constituted from a silicate of the formula (SiO₂)ₙ(Na₂O)ₘ(K₂O)ₚ, where n denotes a positive rational number and m and p, mutually independently, denote a positive rational number or 0, with the provisions that at least one of the parameters m or p is different from 0, and the ratio between n and the sum of m and p is between 2:1 and 4:1.

The solid cosmetic carriers can furthermore contain so-called pouring aids, which are intended to prevent clumping or caking of the powder constituents. Preferred appropriate pouring aids of this kind are water-insoluble, hydrophobizing, or moisture-absorbing powders of diatomaceous earth, pyrogenic silicic acids, calcium phosphate, calcium silicates, aluminum oxide, magnesium oxide, magnesium carbonate, zinc oxide, stearates, fatty amines, and the like.

Lastly, the solid cosmetic carriers can also additionally contain a dedusting agent that prevents the powdered constituents from forming dust. Inert oils, in particular, can be used for this. The solid cosmetic carriers preferably contain ester oils or mineral oils, preferably hydrocarbon oils such as liquid paraffin oil, as a dedusting agent.

Preparation (A) contains at least one persulfate as a first essential ingredient. Persulfates suitable according to the present invention are inorganic peroxo compounds. These are preferably selected from ammonium peroxodisulfate, alkali metal peroxodisulfates, ammonium peroxomonosulfate, alkali metal peroxomonosulfates, alkali metal peroxodiphosphates, and/or alkaline earth metal peroxides. Ammonium peroxodisulfate and/or alkali metal peroxodisulfates are particularly preferred.

In a preferred embodiment of the present invention, preparation (A) contains as a persulfate at least one peroxodisulfate salt, in particular selected from ammonium peroxodisulfate and/or potassium peroxodisulfate and/or sodium peroxodisulfate.

It has also proven, in the context of the work toward the present invention, to be particularly preferred if preparations (A) contain at least two different peroxodisulfates. Preferred peroxodisulfate salts in this context are combinations of ammonium peroxodisulfate with potassium peroxodisulfate and/or sodium peroxodisulfate.

Preparations (A) contain persulfate salts by preference in a quantity from 0.1 to 80 wt %, preferably from 2 to 50 wt %, particularly preferably from 3 to 30 wt %, and especially preferably 5 to 15 wt %, explicitly 5, 6, 7, 8, 9, 10, 11, 12, 13, 14, or 15 wt %, based in each case on the total weight of the agent.

A further embodiment of the present invention is characterized in that preparation (A) is free of xanthan. “Free of” is to be understood for purposes of the invention as quantities of 0.01 wt % and less, based on preparation (A).

The first subject of the invention encompasses agents for lightening keratinic fibers that contain at least two preparations (A) and (B) packaged separately from one another, as well as optionally a further preparation (C) packaged separately from (A) and (B). Preparations (B) and optionally (C) contain the active substances in a flowable cosmetic carrier. The basis of the flowable cosmetic carrier is preferably aqueous or aqueous alcoholic. For purposes of hair bleaching, such carriers are, for example, transparent gels or also surfactant-containing foaming solutions such as, for example, shampoos, foam aerosols, or other preparations that are suitable for utilization on the hair A preferred flowable carrier contains, for purposes of the invention, at least 40 wt %, in particular at least 50 wt % water. “Aqueous alcoholic” carriers are to be understood for purposes of the present invention as water-containing compositions containing 3 to 70 wt % of a C₁ to C₄ alcohol, in particular ethanol resp. isopropanol. The agents according to the present invention can additionally contain further organic solvents, for example methoxybutanol, ethyl diglycol, 1,2-propylene glycol, n-propanol, n-butanol, n-butylene glycol, glycerol, diethylene glycol monoethyl ether, and diethylene glycol mono-n-butyl ether. All water-soluble organic solvents are preferred in this context.

Preparations (B) and/or preparations (C) of the hair-bleaching agent in accordance with the invention contain at least one natural polymer as an essential ingredient.

If the agent for lightening keratinic fibers contains exactly two preparations (A) and (B) packaged separately from one another, which are mixed immediately before utilization to yield a utilization mixture, preparation (B) in accordance with the invention then contains at least one natural polymer.

If the agent for lightening keratinic fibers contains at least three preparations (A), (B), and (C) packaged separately from one another, which are mixed immediately before utilization to yield a utilization mixture, preparation (B) and/or preparation (C) can then contain at least one natural polymer.

Cellulose derivatives that are utilized as thickening agents can be used, for example, as a natural polymer. Examples are agar-agar, carrageenan, alginates, xanthan gum, karaya gum, ghatti gum, tragacanth, scleroglucan gums, or gum arabic, alginates, pectins, polyoses, guar gums, locust bean flour, linseed gums, dextrans, pectins, starch fractions and derivatives such as amylose, amylopectin, and dextrins, gelatins, and casein, as well as cellulose derivatives such as, for example, methyl cellulose, carboxyalkyl celluloses such as carboxymethyl cellulose, and hydroxyalkyl celluloses such as hydroxyethyl cellulose.

Natural polymers from the substance classes recited are commercially obtainable and are offered, for example, under the commercial names Deuteron®-XG (anionic heteropolysaccharide based on β-D-glucose, D-manose, D-glucuronic acid, Schoener GmbH), Deuteron®-XN (nonionogenic polysaccharide, Schoener GmbH), Protanal RF 6650 alginate (sodium alginate, FMC Biopolymer), Cekol (cellulose gum, Kelco), Kelzan (xanthan biopolymer, Kelco), Xanthan FN (xanthan biopolymer, Jungbunzlauer), Keltrol, e.g. Keltrol CG-T (xanthan biopolymer, Kelco) or Keltrol CG-SFT (xanthan biopolymer, Kelco).

In a preferred embodiment of the invention, preparations (B) and/or optionally (C) contain xanthan.

Those xanthans that yield transparent preparations after swelling are preferred according to the present invention. It is particularly preferred to use the xanthan biopolymer that is marketed under the trade name Keltrol CG-SFT of the Kelco company.

In a preferred embodiment, preparation (B) contains, based on its weight, xanthan in quantities from 0.1 to 10 wt %, by preference from 0.5 to 6 wt %, particularly preferably from 0.7 to 5 wt %, and especially preferably from 1 to 4 wt %, explicitly 1, 2, 3, or 4 wt %, if the agent for lightening keratinic fibers contains exactly two preparations (A) and (B) packaged separately from one another, which are mixed immediately before utilization to yield a utilization mixture. The completely mixed utilization preparations contain, based on their weight, xanthan in quantities by preference from 0.6 to 5 wt %, particularly preferably from 1.0 to 3.5 wt %, and especially preferably from 1.5 to 2.5 wt %, explicitly 1.5, 1.6, 1.7, 1.8, 1.9, 2.0, 2.1, 2.2, 2.3, 2.4, or 2.5 wt %.

If the agent for lightening keratinic fibers contains at least three preparations (A), (B), and (C) packaged separately from one another, which are mixed immediately before utilization to yield a utilization mixture, preparation (B) and/or preparation (C) can then contain xanthan. If the agent for lightening keratinic fibers contains at least three preparations (A), (B), and (C) packaged separately from one another, which are mixed immediately before utilization to yield a utilization mixture, preparations in which preparation (C) contains xanthan are preferred. Preparations in which preparation (C) contains xanthan and preparation (B) is free of xanthan are particularly preferred.

Regardless of whether preparation (B) and/or preparation (C) contain xanthan, those completely mixed utilization preparations which contain, based on their weight, xanthan in quantities by preference from 0.6 to 5 wt %, particularly preferably from 1.0 to 3.5 wt %, and especially preferably from 1.5 to 2.5 wt %, explicitly 1.5, 1.6, 1.7, 1.8, 1.9, 2.0, 2.1, 2.2, 2.3, 2.4, or 2.5 wt %, are preferred.

In a particular embodiment, preparations (B) in accordance with the invention contain hydrogen peroxide as an oxidizing agent.

The concentration of a hydrogen peroxide solution in the oxidizing agent preparation (B) is determined on the one hand by regulatory provisions and on the other hand by the desired effect. Preparations (B) contain, based on their weight, hydrogen peroxide in quantities by preference from 0.5 to 30 wt %, preferably from 1 to 20 wt %, particularly preferably from 5 to 15 wt %, and especially preferably 6 to 12 wt %, explicitly 6, 7, 8, 9, 10, 11, or 12 wt %.

Ready-to-use agents preferred according to the present invention are characterized in that they contain, based on the total weight of the ready-to-use agent, 0.01 to 12 wt %, by preference 0.1 to 10 wt %, particularly preferably 1 to 8 wt % hydrogen peroxide.

To stabilize the hydrogen peroxide, the pH of preparation (B) can be adjusted preferably to pH 3 to 5, particularly preferably to pH 3.5 to 4.5, and especially preferably to pH 3.8 to 4.2.

The viscosity properties of preparation (B) are of significance for good miscibility and stability thereof. In a preferred embodiment, preparations (B) of the present invention are characterized in that they have a viscosity from 1000 mPa·s to 50,000 mPa·s, by preference from 5000 mPa·s to 45,000 mPa·s, and particularly preferably from 7000 mPa·s to 40,000 mPa's, in measurements with a Brookfield rotary viscosimeter, spindle size 4, at 25° C. and 4 rpm. The completely mixed and ready-to-use agents have a viscosity preferably from 10,000 mPa·s to 50,000 mPa·s, and particularly preferably from 18,000 mPa·s to 30,000 mPa·s, in measurements with a Brookfield rotary viscosimeter, spindle size 5, at 25° C. and 4 rpm.

Adjustment of the pH is furthermore of significance for good miscibility and stability. Completely mixed and ready-to-use agents whose pH is between 9 and 12 are preferred according to the present invention.

It can also be advantageous according to the present invention if preparation (B) contains at least one nonionic surfactant, preferably at least one ethoxylated fatty alcohol with 40 to 60 ethylene oxide units. This is to be understood according to the present invention as an addition product of ethylene oxide with a fatty alcohol. Fatty alcohols are, in this context, saturated and unsaturated alcohols having 12 to 24 carbon atoms, which can be linear or branched. The molar quantity of ethylene oxide that was used per mol of fatty alcohol is understood to designate the degree of ethoxylation. Suitable in this context as a nonionic surfactant are, in particular, ethylene oxide addition products with octyl alcohol (capryl alcohol), nonyl alcohol (pelargonyl alcohol), undecyl alcohol, undec-10-en-1-ol, dodecyl alcohol (lauryl alcohol), 2,6,8-trimethyl-4-nonanol (isolauryl alcohol), tridecyl alcohol, tetradecyl alcohol (myristyl alcohol), pentadecyl alcohol, hexadecyl alcohol (cetyl/palmityl alcohol), heptadecyl alcohol, octadecyl alcohol (stearyl alcohol), isostearyl alcohol, (9Z)-octadec-9-en-1-ol (oleyl alcohol), (9E)-octadec-9-en-1-ol (elaidyl alcohol), (9Z,12Z)-octadeca-9,12-dien-1-ol (linoleyl alcohol), (9Z ,12Z ,15Z)-octadeca-9,12,15-trien-1-ol (linolenyl alcohol), nonadecan-1-ol (nonadecyl alcohol), eicosan-1-ol (eicosyl alcohol/arachyl alcohol), (9Z)-eicos-9-en-1-ol (gadoleyl alcohol), (5Z,8Z,11Z,14Z)-eicosa-5,8,11,14-tetraen-1-ol (arachidonyl alcohol), heneicosyl alcohol, docosyl alcohol (behenyl alcohol), (13Z)-docos-13-en-1-ol (erucyl alcohol), or (13E)-docosen-1-ol (brassidyl alcohol). It is likewise possible according to the present invention to utilize mixtures of fatty alcohols that occur by deliberate mixing or also as a result of recovery methods. Examples are coconut alcohol (mixture of C₈ to C₁₈ fatty alcohols) or cetearyl alcohol (1:1 mixture of C₁₆ and C₁₈ fatty alcohols).

Degrees of ethoxylation from 20 to 60 are preferred. Nonionic surfactants of the ethoxylated fatty alcohol type that are preferred according to the present invention are ceteareth-20 and ceteareth-50.

The hair-bleaching agents can furthermore contain alkalizing agents. Preferred alkalizing agents are, for example, ammonia, alkanolamines, basic amino acids, as well as inorganic alkalizing agents such as alkali/alkaline earth metal hydroxides, alkali/alkaline earth metal metasilicates, alkali/alkaline earth metal phosphates, and alkali/alkaline earth metal hydrogen phosphates. Lithium, sodium, and/or potassium preferably serve as metal ions. A particularly preferred alkalizing agent is ammonia.

Inorganic alkalizing agents usable according to the present invention are preferably selected from sodium hydroxide, potassium hydroxide, calcium hydroxide, barium hydroxide, sodium phosphate, potassium phosphate, sodium silicate, potassium silicate, magnesium silicate, sodium carbonate, and potassium carbonate. Sodium hydroxide and/or potassium hydroxide are particularly preferred.

Alkalizing agents usable according to the present invention are preferably selected from alkanolamines of primary, secondary, or tertiary amines having a C₂ to C₆ alkyl basic structure that carries at least one hydroxyl group. Particularly preferred alkanolamines are selected from the group that is constituted from 2-aminoethan-1-ol (monoethanolamine), 3 -aminopropan-1-ol, 4 -aminobutan-1-ol, 5 -aminopentan-1-ol, 1-aminopropan-2-ol (monoisopropanolamine), 1-aminobutan-2-ol, 1-aminopentan-2-ol, 1-aminopentan-3-ol, 1-aminopentan-4-ol, 2-amino-2-methylpropanol, 2-amino-2-methylbutanol, 3-amino-2-methylpropan-1-ol, 1-amino-2-methylpropan-2-ol, 3-aminopropane-1,2-diol, 2-amino-2-methylpropane-1,3-diol, 2-amino-2-ethyl-1,3-propanediol, N,N-dimethylethanolamine, methylglucamine, triethanolamine, diethanolamine, and triisopropanolamine. Particularly preferred alkanolamines are monoethanolamine, 2-amino-2-methylpropanol, and triethanolamine.

The basic amino acids usable as an alkalizing agent according to the present invention are preferably selected from the group that is constituted from L-arginine, D-arginine, D/L-arginine, L-lysine, D-lysine, D/L-lysine, L-omithine, D-ornithine, D/L-ornithine, L-histidine, D-histidine, and/or D/L-histidine. L-arginine, D-arginine, and/or D/L-arginine are particularly preferably used as an alkalizing agent for purposes of the invention.

Some consumers find the intense odor produced by ammonia to be annoying or irritating. Although ammonia is a preferred alkalizing agent. ready-to-use preparations that are free of ammonia can therefore be preferred according to the present invention. Preferred alkalizing agents for preparations that are free of ammonia are monoethanolamine, 2-amino-2-methypropanol, and triethanolamine.

If the ready-to-use mixtures contain alkalizing agents, preparations that contain alkalizing agents in a quantity from 0.05 to 20 wt %, in particular from 0.5 to 10 wt %, based in each case on the total weight of the ready-to-use agent, are preferred according to the present invention.

In a further embodiment of the invention, preparations (A) and (B) can be mixed with further separately packaged preparations immediately before utilization to yield a utilization mixture.

In a preferred embodiment of the invention, the agent according to the present invention additionally contains at least one further preparation (C) packaged separately from preparations (A) and (B), preparation (C) containing at least one alkalizing agent and at least one natural polymer.

Preparation (C) preferably contains natural polymers that have already been recited earlier in the text in conjunction with preparation (B).

Those alkalizing agents that have already been described earlier are preferred according to the present invention.

If preparations (C) contain alkalizing agents, preparations that contain alkalizing agents in a quantity from 0.05 to 20 wt %, in particular from 0.5 to 10 wt %, based in each case on the total weight of the ready-to-use agent, are preferred according to the present invention.

Regardless of whether preparations (C) and/or preparation (B) and/or further preparations contain alkalizing agents, if alkalizing agents are used, those preparations that contain alkalizing agents in a quantity from 0.05 to 20 wt %, in particular from 0.5 to 10 wt %, based in each case on the total weight of the ready-to-use agent, are preferred according to the present invention.

To further enhance the lightening performance, a silicon-containing compound can additionally be added to preparation (C) as a bleach intensifier. Said compound is preferably selected from the group that is constituted from silicic acid, alkali metal silicates, and alkaline earth metal silicates.

Although even small quantities of the silicon-containing compounds already increase lightening performance, it can be preferred according to the present invention to use the silicon-containing compounds in quantities from 0.05 wt % to 50 wt %, preferably in quantities from 0.5 wt % to 30 wt %, and particularly preferably in quantities from 1.0 wt % to 25 wt %, based in each case on the total weight of preparation (C).

Alkali metal silicates in the form of water glass are used, in particular, as silicon-containing compounds. “Water glass” is to be understood here as a compound that is constituted from a silicate of the formula (SiO₂)ₙ(Na₂O)ₘ(K₂O)ₚ, where n denotes a positive rational number and m and p, mutually independently, denote a positive rational number or 0, with the provisions that at least one of the parameters m or p is different from 0, and the ratio between n and the sum of m and p is between 1:1 and 4:1.

In addition to the components described by the empirical formula, the water glasses can also contain further additives in small quantities, such as phosphates or magnesium salts.

Water glasses that are particularly preferred according to the present invention are marketed, inter alia, under the designations Ferrosil® 119, Natronwasserglas 40/42, Portil® A, Portil® AW and Portil® W, and Britesil® C20.

In addition, silicic acids in particular, which are also marketed as “silica” or “silica gel,” can be used as silicon-containing compounds. A silica gel that is marketed under the commercial name Aerosil 200 (INCI name: Silica) is preferred in this context.

The lightening or hair-bleaching agents can further contain additional bleaching power intensifiers in order to intensify the hair-bleaching effect. Compounds that, under perhydrolysis conditions, yield aliphatic peroxocarboxylic acids having by preference 1 to 10 carbon atoms, in particular 2 to 4 carbon atoms, and/or (optionally substituted) perbenzoic acid, can be used as bleach intensifiers. Multiply acylated alkylenediamines, in particular tetraacetylethylendiamine (TAED), acylated triazine derivatives, in particular 1,5-diacetyl-2,4-dioxohexahydro-1,3,5-triazine (DADHT), acylated glycolurils, in particular tetraacetyl glycoluril (TAGU), N-acylimides, in particular N-nonanoyl succinimide (NOSI), acylated phenolsulfonates, in particular n-nonanoyl or isononanoyl oxybenzenesulfonate (n- resp. iso-NOBS), carboxylic acid anhydrides, in particular phthalic acid anhydride, acylated polyvalent alcohols, in particular triacetin, ethylene glycol diacetate, and 2,5-diacetoxy-2,5-dihydrofuran, are preferred.

In preferred fashion according to the present invention, carbonate salts resp. hydrogen carbonate salts can be used as bleach intensifiers of the carbonic-acid derivative type. These are preferably selected from the group of the ammonium, alkali metal (in particular potassium and sodium), and alkaline earth metal (in particular magnesium and calcium) carbonate salts resp. hydrogen carbonate salts. Particularly preferred carbonate resp. hydrogen carbonate salts are ammonium hydrogen carbonate, ammonium carbonate, sodium hydrogen carbonate, sodium carbonate, potassium hydrogen carbonate, potassium carbonate, magnesium carbonate, and calcium carbonate. These particularly preferred salts can be used as bleach intensifiers alone, or in mixtures of at least two representatives thereof. Alkyl carbonates, alkyl carbamates, and silyl carbonates and silyl carbamates are also suitable bleach intensifiers.

Bleaching power intensifiers usable according to the present invention can furthermore be selected from nitrogen-containing, optionally cationic heterocycles, in particular imidazole.

Particularly preferred nitrogen-containing heterocyclic bleaching power intensifiers are the quaternized cations of pyridines and of 3,4-dihydroisoquinolines, such as salts of 4-acetyl-1-methylpyridinium, in particular 4-acetyl-1-methylpyridinium-p-toluenesulfonate, salts of 2-acetyl-1-methylpyridinium, in particular 2-acetyl-1-methylpyridinium-p-toluenesulfonate, and salts of N-methyl-3,4-dihydroisoquinolinium, in particular N-methyl-3,4-dihydroisoquinolinium-p-toluenesulfonate.

Urea is also a bleaching power intensifier usable according to the present invention.

Bleaching power intensifiers can be contained in preparation (A) and/or preparation (B) and/or optionally preparation (C) and/or optionally further preparations. The bleaching power intensifiers can be contained either in only one of the preparations or in two or more of the preparations. Hydrolysis-sensitive bleaching power intensifiers can preferably be used in the powdered preparation (A). Regardless of whether bleaching power intensifiers are used in preparation (A) and/or preparation (B) and/or preparation (C) and/or further preparations, they are contained, if bleaching power intensifiers are utilized, preferably in quantities from 0.5 to 30 wt %, in particular in quantities from 2 to 20 wt %, based in each case on the total weight of the completely mixed hair-bleaching preparation.

In addition, to suppress undesired residual color impressions especially in the reddish or bluish region, the lightening resp. hair-bleaching agents can contain specific substantive dyes of the complementary colors. These are dyes that absorb directly onto the hair and do not require an oxidizing process in order to form the color. Substantive dyes are usually nitrophenylenediamines, nitroaminophenols, azo dyes, anthraquinones, or indophenols. Substantive dyes are known as anionic, cationic, and nonionic substantive dyes. The substantive dyes are respectively used preferably in a quantity from 0.001 to 2 wt %, based on the entire utilization preparation.

Preferred anionic substantive dyes are the compounds known under the international designations resp. commercial names Acid Yellow 1, Yellow 10, Acid Yellow 23, Acid Yellow 36, Acid Orange 7, Acid Red 33, Acid Red 52, Pigment Red 57:1, Acid Blue 7, Acid Green 50, Acid Violet 43, Acid Black 1, Acid Black 52, bromophenol blue, and tetrabromophenol blue. Preferred cationic substantive dyes are cationic triphenylmethane dyes such as, for example, Basic Blue 7, Basic Blue 26, Basic Violet 2, and Basic Violet 14, aromatic systems that are substituted with a quaternary nitrogen group, for example Basic Yellow 57, Basic Red 76, Basic Blue 99, Basic Brown 16, and Basic Brown 17, cationic anthraquinone dyes such as HC Blue 16 (Bluequat B), as well as substantive dyes which contain a heterocycle that comprises at least one quaternary nitrogen atom, in particular Basic Yellow 87, Basic Orange 31, and Basic Red 51. The cationic substantive dyes that are marketed under the Arianor trademark are likewise preferred cationic substantive dyes according to the present invention. Nonionic nitro and quinone dyes, and neutral azo dyes, are particularly suitable as nonionic substantive dyes. Preferred nonionic substantive dyes are the compounds known under the international designations resp. commercial names HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9, as well as 1,4-diamino-2-nitrobenzene, 2-amino-4-nitrophenol, 1,4-bis-(2-hydroxyethyl)amino-2-nitrobenzene, 3-nitro-4-(2-hydroxyethyl)aminophenol, 2-(2-hydroxyethyl)amino-4,6-dinitrophenol, 4-[(2-hydroxyethyl)amino]-3 -nitro-1-methylbenzene, 1-amino-4-(2-hydroxyethyl)amino-5-chloro-2-nitrobenzene, 4-amino-3-nitrophenol, 1-(2′-ureido ethyl)amino-4-nitrobenzene, 2-[(4-amino-2-nitrophenyl)amino]benzoic acid, 6-nitro-1,2,3,4-tetrahydroquinoxaline, 2-hydroxy-1,4-naphthoquinone, picramic acid and salts thereof, 2-amino-6-chloro-4-nitrophenol, 4-ethylamino-3-nitrobenzoic acid, and 2-chloro-6-ethylamino-4-nitrophenol. Agents that contain at least one combination of tetrabromophenol blue and Acid Red 92 are very particularly preferred.

It has further proven to be advantageous if the ready-to-use agents contain at least one stabilizer or complexing agent. Particularly preferred stabilizers are phenacetin, alkali benzoates (sodium benzoate), and salicylic acid. All complexing agents of the existing art can furthermore be used. These can belong to different chemical groups. By preference, they are used individually or mixed with one another. Complexing agents preferred according to the present invention are nitrogen-containing polycarboxylic acids, in particular EDTA, and phosphonates, by preference hydroxyalkane- resp. aminoalkanephosphonates, and in particular 1-hydroxyethane-1,1-diphosphonate (HEDP) resp. the di- or tetrasodium salt thereof, and/or ethylenediaminetetramethylenephosphonate (EDTMP) resp. the hexasodium salt thereof, and/or diethylenetriaminepentamethylenephosphonate (DTPMP) resp. the hepta- or octasodium salt thereof.

The agents according to the present invention can moreover contain further active substances, adjuvants, and additives such as, for example, nonionic polymers such as, for example, vinylpyrrolidinone/vinyl acrylate copolymers, polyvinylpyrrolidinone, vinylpyrrolidinone/vinyl acetate copolymers, polyethylene glycols, and polysiloxanes; additional silicones such as volatile or nonvolatile straight-chain, branched, or cyclic, crosslinked or uncrosslinked polyalylsiloxanes (such as dimethicones or cyclomethicones), polyarylsiloxanes and/or polyalkylarylsiloxanes, in particular polysiloxanes having organofunctional groups, such as substituted or unsubstituted amines (amodimethicones), carboxyl, alkoxy, and/or hydroxyl groups (dimethicone copolyols), linear polysiloxane(A)-polyoxyalkylene(B) block copolymers, grafted silicone polymers; cationic polymers such as quaternized cellulose ethers, polysiloxanes having quaternary groups, dimethyldiallylammonium chloride polymers, acrylamide/dimethyldiallylammonium chloride copolymers, dimethylaminoethyl methacrylate/vinylpyrrolidinone copolymers quaternized with diethyl sulfate, vinylpyrrolidinone/imidazolinium methochloride copolymers, and quaternized polyvinyl alcohol; zwitterionic and amphoteric polymers; anionic polymers such as, for example, polyacrylic acids or crosslinked polyacrylic acids; structuring agents such as glucose, maleic acid, and lactic acid, hair-conditioning compounds such as phospholipids, for example lecithin and kephalins; perfume oils, dimethylisosorbide, and cyclodextrins; fiber-structure-improving active substances, in particular mono-, di- and oligosaccharides such as, for example, glucose, galactose, fructose, fruit sugars, and lactose; dyes for coloring the agent; anti-dandruff active substances such as piroctone olamine, zinc omadine and climbazol; amino acids and oligopeptides, in particular arginine and/or serine; animal- and/or plant-based protein hydrolysates such as, for example, protein hydrolysates of elastin, collagen, keratin, silk, and milk, or protein hydrolysates of almond, rice, bean, potato, and wheat, as well as derivatives in the form of fatty acid condensation products thereof or optionally anionically or cationically modified derivatives; light-protection agents, such as derivatized benzophenones, cinnamic acid derivatives, and triazines; active substances such as panthenol, pantothenic acid, pantolactone, allantoin, pyrrolidinonecarboxylic acids and salts thereof, as well as bisabolol; polyphenols, in particular hydroxycinnamic acids, 6,7-dihydroxycumarins, hydroxybenzoic acids, catechins, tannins, leucoanthocyanidines, anthocyanidines, flavanones, flavones, and flavonols; ceramides or pseudoceramides; vitamins, provitamins, and vitamin precursors, in particular those of the groups A, B₃, B₅, B₆, C, E, F, and H; plant extracts; swelling and penetration substances such as glycerol, propylene glycol monoethyl ether, carbonates, hydrogen carbonates, guanidines, ureas, and primary, secondary, and tertiary phosphates; luster agents such as ethylene glycol mono- and distearate as well as PEG-3 distearate; pigments, and propellants such as propane/butane mixtures, N₂O, dimethyl ether, CO₂, and air.

Particularly preferred according to the invention are those active substances, adjuvants, and additives that result, in combination with the agent according to the present invention, in a transparent utilization mixture.

One skilled in the art will arrive at a selection of these further substances in accordance with the desired properties of the agents. With regard to further optional components, as well as the quantities of those components used, reference is made expressly to the relevant manuals known to one skilled in the art, e.g. Kh. Schrader, Grundlagen and Rezepturen der Kosmetika [Cosmetics fundamentals and formulations], 2nd ed., Hiithig Buch Verlag, Heidelberg, 1989. The additional active substances and adjuvants are used respectively in the agents according to the present invention preferably in quantities from 0.0001 to 10 wt %, in particular from 0.0005 to 5 wt %, based on the total weight of the utilization mixture.

A second subject of the invention is a method for changing the color of keratinic fibers, wherein at least two preparations (A) and (B) packaged separately from one another, of which preparation (A) contains at least one persulfate and preparation (B) at least one oxidizing agent and at least one natural polymer, are mixed to yield a utilization mixture, the latter is applied onto the fibers, and is rinsed out again after a contact time.

The ready-to-use agents are produced, immediately before utilization on the hair, by mixing the two preparations (A) and (B) and optionally a third preparation (C) and/or further preparations. In the case of ready-to-use agents that are mixed from more than two preparations to yield a completed utilization mixture, it can be immaterial whether firstly two preparations are mixed with one another and then the third preparation is added and mixed in, or whether all the preparations are combined together and then mixed. Mixing can be accomplished by stirring in a dish or cup, or by shaking in a closable container.

The term “immediately” is to be understood as a time period from a few seconds to one hour, by preference up to 30 minutes, in particular up to 15 minutes.

The agents according to the present invention are utilized in a method for lightening keratinic fibers, in particular human hair, in which the agent is applied onto the keratin-containing fibers, left on the fibers at a temperature from room temperature to 45° C. for a contact period from 10 to 60 minutes, and then rinsed out again with water or washed out with a shampoo.

The contact time of the ready-to-use lightening agents is preferably 10 to 60 minutes, in particular 15 to 50 minutes, particularly preferably 20 to 45 minutes. During the contact time of the agent on the fibers, it can be advantageous to assist the lightening operation by delivering heat. Heat delivery can occur by way of an external heat source, for example using a warm air blower, and also, in particular in the case of a hair lightening process on living subjects, by way of the body temperature of the subject. With the latter option, the portion to be lightened is usually covered with a hood. A contact phase at room temperature is likewise in accordance with the present invention. The temperature during the contact time is preferably between 20° C. and 40° C., in particular between 25° C. and 38° C. The lightening agents already produce good hair-bleaching and lightening results at physiologically acceptable temperatures of less than 45° C.

After the contact time has ended, the remaining lightening preparation is rinsed out of the hair with water or with a cleaning agent. A commercially usual shampoo can, in particular, serve as a cleaning agent in this context, in which case in particular the cleaning agent can then be omitted and the rinsing-out operation can occur using tap water if the lightening agent possesses a carrier with a high surfactant content.

A preferred method is characterized in that monitoring of the degree of lightening of the keratinic fibers during the contact time occurs visually, without removing the utilization mixture from the fibers. For this, a ready-to-use transparent agent of the first subject of the invention is applied onto human hair, and the lightening process is assessed during the contact time, once or several time, by visual monitoring, without removing the agent from the fibers. This ensures simplified and continuous monitoring of the decolorizing operation.

Preparations that, upon application of a uniform layer of said preparations from 1 to 3 mm in thickness onto a substrate, are clear, and can be seen through in such a way that the human eye can detect and assess the color of the substrate without cloudiness, are “transparent” for purposes of the invention. Transparency can furthermore be measured by one skilled in the art using technical methods. Preparations that, in photometric measurements with a Methrom 662 photometer at 25° C., achieve transmittances of at least 70%, in particular at least 80%, are therefore also “transparent” for purposes of the invention.

The preferred embodiments of the first subject of the invention also apply, mutatis mutandis, to the second subject of the invention.

A third subject of the present invention is a method for manufacturing a flowable oxidizing agent preparation that contains at least one oxidizing agent, wherein powdered xanthan gum is stirred into the preparation, which gum has the property that 1 wt % of the xanthan gum in a 1-wt % aqueous KCl solution exhibits a viscosity from 800 to 1300 mPa·s in measurements with a Brookfield rotary viscosimeter, spindle size 4, at 25° C. and 4 rpm.

The xanthan gum used is by preference present in a powdered state prior to swelling, and is characterized in that the powdered xanthan has a particle size of between 160 μm and 200 μm.

The manner of packaging the lightening agents according to the present invention is in principle subject to no limitations whatsoever. In order to offer the components of the ready-to-use agent to the user as conveniently as possible, it is useful to market the individual preparations together in one packaging unit.

A fourth subject of the present invention is therefore a multi-component packaging unit (kit of parts) for lightening and/or hair-bleaching of keratinic fibers, encompassing, each in containers packaged separately from one another:
a) at least one powdered preparation (A) containing at least one persulfate,
b) at least one flowable preparation (B) containing at least one oxidizing agent and at least one natural polymer,
c) optionally, a further preparation (C) containing at least one alkalizing agent and at least one natural polymer,
   and/or optionally further preparations.

The components of the multi-package unit are packaged separately from one another in physically different containers. The term “container” characterizes, in this context, a receiving capability, regardless of its shape, material, or closure, which embodies the capability of containing substances or substance mixtures. The term “container” therefore encompasses, without being limited thereto, the interior of a tube, of a pouch or sack, of a canister, of a tin, of a pan, of a bottle, of a glass or a packet, of a carton, of a box, of an envelope, or of another receptacle. The containers can be equipped with a reclosable opening such as a screw closure. This can be advantageous in particular when multiple agents are to be intimately mixed with one another, by shaking, before use.

The components of the lightening preparation can be contained in a double-chamber container having a separate or shared opening. It is preferred, however, to distribute them among different containers and to instruct the consumer to mix them with one another before utilization.

The multi-component packaging unit (kit of parts) preferably additionally contains a set of instructions for use. The instructions for use contain, in particular, information, explanations, and optionally illustrations for the user for utilizing the agents from the containers of the packaging unit in a method in accordance with the second subject of the invention. It may furthermore be preferred if a mixing aid, for example a dish, an application aid, for example a comb or brush, and/or personal protection equipment, for example disposable gloves, are included with the kit.

With regard to preferred embodiments of the multi-component packaging unit according to the present invention, the embodiments of the foregoing subjects of the invention apply mutatis mutandis.

### EXAMPLES

### Example 1

### Composition of Preparation (A) for Hair-Bleaching Agents Made Up of Two Preparations

| Description | A1 | A2 | A3 | A4 | A5 | A6 | A7 | A8 |
|---|---|---|---|---|---|---|---|---|
| EDTA Na2 | 1.51 | 1 | 1.51 | 1.51 | 1.51 | 1.51 | 1.51 | 1.51 |
| Sodium hexametaphosphate | 0.3 | 0.3 | 0.3 | 0.3 | 0.3 | 0.3 | 0.3 | 0.3 |
| Aerosil 200 | 3.02 | 1 | 3 | 3 | 3 | 3 | 3 | 3 |
| Sodium metasilicate FE, | 26 | 26 | 26 | 26 | 26 | 26 | 26 | 26 |
| anhydrous | | | | | | | | |
| Calcium stearate | 0.9 | 0.9 | 0.9 | 0.9 | 0.9 | 0.9 | 0.9 | 0.9 |
| Rohagit S hv | 3.02 | 4.05 | 1 | | | 2.5 | 3.5 | |
| Cekol 50000 | 5 | 6.5 | 2.5 | 2.5 | 2.5 | 1 | | |
| Ammonium persulfate + | 19.9 | 19.9 | 19.9 | 19.9 | 19.9 | 19.9 | 19.9 | 19.9 |
| 0.5% silicic acid | | | | | | | | |
| Potassium persulfate | 7.4 | 7.4 | 11.94 | 11.94 | 11.94 | 11.94 | 11.94 | 15.44 |
| Sodium persulfate | 24.9 | 24.9 | 24.9 | 24.9 | 24.9 | 24.9 | 24.9 | 24.9 |
| Paraffinum Liquidum | 7.85 | 7.85 | 7.85 | 7.85 | 7.85 | 7.85 | 7.85 | 7.85 |
| Perfume: ROSEX FINE INC | 0.2 | 0.2 | 0.2 | 0.2 | 0.2 | 0.2 | 0.2 | 0.2 |
| 637510 | | | | | | | | |
| Laponite XLG | | | | 1 | | | | |
| Laponite XLS | | | | | 1 | | | |

*Raw materials used: Aerosil 200 (INCI name: Silica (Evonik Degussa)), Rohagit S hv (INCI name: Acrylates Copolymer (Evonik Röhm)), Cekol 50000 (INCI name: Cellulose Gum (CP Kelco)), Laponite XLG (INCI name: Sodium Magnesium Silicate (Rockwood Additives)), Laponite XLS (INCI name: Sodium Magnesium Silicate and Tetrasodium Pyrophosphate (Rockwood Additives)).

### Example 2

### Composition of Preparation (B) for Hair-Bleaching Agents Made Up of Two Preparations

| Description | B1 | B2 | B3 | B4 | B5 |
|---|---|---|---|---|---|
| Water, deionized | to 100 | to 100 | to 100 | to 100 | to 100 |
| Caustic soda 45%, techn. | 0.73 | 0.73 | 0.73 | 0.73 | 0.73 |
| Dipicolinic acid | 0.1 | 0.1 | 0.1 | 0.1 | 0.1 |
| Disodium pyrophosphate | 0.03 | 0.03 | 0.03 | 0.03 | 0.03 |
| HEDP 60% | 1.5 | 1.5 | 1.5 | 1.5 | 1.5 |
| Keltrol CG-SFT | 2 | | 1.5 | 1 | 0.5 |
| Propanediol-1,2 | 4 | 2 | 4 | 4 | 4 |
| Hydrogen peroxide 50% | 18 | 18 | 18 | 18 | 18 |
| Xanthan FN | | 1 | | | |

* Raw materials used: Keltrol CG-SFT (INCI name: Xanthan Gum (CP Kelco)), Xanthan FN (INCI name: Xanthan Gum (Jungbunzlauer)).

Preparations B1 to B5 are transparent gels.

### Example 3

### Composition of Preparation (A) for Hair-Bleaching Agents Made Up of Three Preparations

| Description | A9 | A10 | A11 |
|---|---|---|---|
| Potassium persulfate | 98.6 | 42.8 | 60 |
| Aerosil 200 | 1.4 | — | — |
| Ammonium persulfate + 4% silicic acid | — | 13 | 18.2 |
| Sodium persulfate | — | 15.6 | 21.8 |
| Britesil C 20 | — | 28 | — |

* Raw materials used: Aerosil 200 (INCI name: Silica (Evonik Degussa)), Britesil C 20 (INCI name: Sodium Silicate (PQ Europe)).

### Example 4

### Composition of Preparation (B) for Hair-Bleaching Agents Made Up of Three Preparations

| Description | B6 | B7 | B8 | B9 | B10 | B11 | B12 |
|---|---|---|---|---|---|---|---|
| Water, deionized | to 100 | to 100 | to 100 | to 100 | to 100 | to 100 | to 100 |
| Caustic soda 45%, techn. | 0.73 | 0.73 | 0.73 | 0.73 | 0.73 | 0.73 | 0.73 |
| Dipicolinic acid | 0.1 | 0.1 | 0.1 | 0.1 | 0.1 | 0.1 | 0.1 |
| Disodium pyrophosphate | 0.03 | 0.03 | 0.03 | 0.03 | 0.03 | 0.03 | 0.03 |
| HEDP 60% | 1.5 | 1.5 | 1.5 | 1.5 | 1.5 | 1.5 | 1.5 |
| Keltrol CG-SFT | 2 | 1.5 | 1 | 0.5 | | 0.5 | |
| Propanediol-1,2 | 4 | 4 | 4 | 4 | | 4 | |
| Hydrogen peroxide 50% | 18 | 18 | 18 | 18 | 18 | 18 | 18 |
| Mergital CS 50 A | | | | | | 10 | 10 |

*Raw materials used: Keltrol CG-SFT (INCI name: Xanthan Gum (CP Kelco)), Mergital CS 50 A (INCI name. Ceteareth-50 (Cognis)).

### Example 5

### Composition of Preparation (C) for Hair-Bleaching Agents Made Up of Three Preparations

| Description | C1 | C2 | C3 |
|---|---|---|---|
| Water, deionized | to 100 | to 100 | to 100 |
| Monoethanolamine | 8 | 8 | 8 |
| Ascorbic acid | 0.4 | 0.4 | 0.4 |
| Sodium sulfite anhydrous A, 96% min. | 0.4 | 0.4 | 0.4 |
| HEDP 60% | 0.2 | 0.2 | 0.2 |
| Keltrol CG-SFT | 2 | 4 | 6 |
| Propanediol-1,2 | 4 | 6 | 8 |
| Sodium water glass | 0.5 | 0.5 | 0.5 |

* Raw materials used: Keltrol CG-SFT (INCI name: Xanthan Gum (CP Keltrol)).

### Examples of Utilization Preparations

### Example 6

### Hair-Bleaching Agent Made Up of Two Preparations

The individual preparations were mixed with one another in the quantities indicated, and the viscosity of the completed utilization preparations was measured with a Brookfield rotary viscosimeter, spindle size 4, at 25° C. and 4 rpm.

| Description | AZ1 | AZ2 | AZ3 | AZ4 | AZ5 | AZ6 | AZ7 | AZ8 |
|---|---|---|---|---|---|---|---|---|
| B5 | 80 g | | | | | | | |
| B4 | | 80 g | | | 80 g | | | |
| B3 | | | 80 g | | | 80 g | | |
| B1 | | | | 80 g | | | 80 g | |
| A8 | 20 g | 20 g | 20 g | 20 g | | | | |
| A1 | | | | | 20 g | 20 g | 20 g | |
| Comp.* | | | | | | | | 120 g* |
| MVisc | 16,300 | 33,100 | 41,500 | 69,700 | 81,300 | 112,000 | 129,000 | 90,750 |
| Sp5/4 rpm | | | | | | | | |
| (mPas) | | | | | | | | |
| Evaluation | clear | clear | clear | clear | clear | clear | clear | white |
| | gel | gel | gel | gel | gel | gel | gel | emulsion |

*Comparison with a product available on the market: 80 g Clynol Viton (9% H₂O), together with 40 g Viton Super Lightening White, yields a white emulsion having a viscosity of 90,750 mPas.

### Exampe 7

### Hair-Bleaching Agents Made Up of Three Preparations

| Description | AZ9 | AZ10 | AZ11 | AZ12 | AZ13 |
|---|---|---|---|---|---|
| B10 | 50 g | 50 g | 40 g | 50 g | |
| B9 | | | | | 50 g |
| C2 | 50 g | 50 g | 40 g | 50 g | 50 g |
| A9 | 8.5 g | | | | 8.5 g |
| A10 | | 20 g | | | |
| A11 | | | 14 g | | |
| MVisc Sp5/ | 35,400 | 38,000 | 37,200 | 37,100 | 46,600 |
| 4 rpm | | | | | |
| (mPas) | | | | | |
| Evaluation | clear gel | clear gel | clear gel | clear gel | clear gel |

The individual preparations were mixed with one another in the quantities indicated, and the viscosity of the completed utilization preparations was measured with a Brookfield rotary viscosimeter, spindle size 5, at 25° C. and 4 rpm.

Utilization preparations AZ 9, AZ 10, AZ 11, and AZ 12 are preparations in accordance with the invention. AZ 9 is an example of a utilization preparation without ammonia.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. Agents for lightening keratinic fibers, comprising at least two preparations (A) and (B) packaged separately from one another, as well as optionally at least one further preparation (C) that is packaged separately from (A) and (B), which are mixed immediately before utilization to yield a utilization mixture, where
i. preparation (A) contains at least one persulfate, and
ii. preparation (B) is flowable and contains at least one oxidizing agent,
wherein at least one of preparation (B) and preparation (C) further comprises at least one natural polymer.

2. The agents according to claim 1, wherein at least one of preparation (B) and preparation (C) comprises xanthan.

3. The agents according to claim 1, wherein preparation (A) is free of xanthan.

4. The agents according to claim 1, wherein preparation (A) comprises at least one persulfate selected from the group consisting of ammonium peroxodisulfate, potassium peroxodisulfate, and sodium peroxodisulfate.

5. The agents according to claim 1, wherein preparation (B) comprises, based on its weight, hydrogen peroxide in quantities from 0.5 to 30 wt %.

6. The agents according to claim 1, wherein preparation (C) is included and comprises at least one alkalizing agent and at least one natural polymer.

7. The agents according to claims 1, wherein the completely mixed utilization mixture comprises, based on its weight, xanthan in quantities from 0.6 to 5 wt %.

8. The agents according to claim 1, wherein the completely mixed utilization preparation has a viscosity from 10,000 mPa·s to 50,000 mPa·s in measurements with a Brookfield rotary viscosimeter, spindle size 5, at 25° C. and 4 rpm.

9. A method for changing the color of keratinic fibers, comprising:
mixing at least two preparations (A) and (B) packaged separately from one another, of which preparation (A) contains at least one persulfate and preparation (B) at least one oxidizing agent and at least one natural polymer, to yield a utilization mixture,
applying the utilization mixture onto the fibers, and
rinsing the fibers out again after a contact time.

10. The method for changing the color of keratinic fibers according to claim 9, further comprising:
visually monitoring the degree of lightening of the keratinic fibers during the contact time without removing the utilization mixture from the fibers.

11. A method for manufacturing a flowable oxidizing agent preparation that comprises at least one oxidizing agent, comprising:
stirring powdered xanthan gum into the preparation, which gum has the property that 1 wt % of the xanthan gum in a 1-wt % aqueous KCl solution exhibits a viscosity from 800 to 1300 mPa·s in measurements with a Brookfield rotary viscosimeter, spindle size 4, at 25° C. and 4 rpm.
